(19) United States
(12) Patent Application Publication
(10) Pub. No.: US 2022/0071255 A1
(43) Pub. Date: Mar. 10, 2022

(54) STRAIN ENGINEERING

(71) Applicant: Radient Technologies Innovations Inc., Edmonton (CA)

(72) Inventors: Michael Cabigon, Edmonton (CA); Jim Seethram, Edmonton (CA); Steven Splinter, Edmonton (CA); Denis Taschuk, Edmonton (CA)

(21) Appl. No.: 17/240,188

(22) Filed: Apr. 26, 2021

Related U.S. Application Data
(63) Continuation of application No. PCT/IB2019/058960, filed on Oct. 22, 2019.
(60) Provisional application No. 62/750,201, filed on Oct. 24, 2018.

(51) Int. Cl. A23L 33/105; A01H 6/28; C12N 15/82

(52) U.S. Cl. CPC A23L 33/105; C12N 15/8243; A01H 6/28

(57) ABSTRACT

Systems and methods for supply chain management are provided that monitor an extraction process through the use of sensors, including optical sensors for hyperspectral analysis or multi-spectral analysis to determine THC and CBD concentrations of the biomass, as well as optical light sensors that track and analyze the extraction operation and correlate extraction results (e.g., amount and quality of concentrate) with specific strains and growing conditions. Such correlation may further be used to improve the efficiency of the plant breeder and grower process by way of feedback regarding what strains and grow conditions are correlated to high quality or high-efficiency extraction results.

### CROSS-REFERENCE TO RELATED APPLICATIONS

The present patent application is a continuation of International Application No. PCT/IB2019/058960 filed Oct. 22, 2019, which claims the priority benefit of U.S. provisional patent application No. 62/750,201 filed Oct. 24, 2018, the disclosures of which are incorporated by reference herein.

### BACKGROUND OF THE INVENTION

### 1. Field of the Technology

The present disclosure is generally related to determining the quality tracking and data correlation of cannabis strains. More specifically, the present disclosure relates to the use of sensors for extraction tracking and data correlation between biomass strain, growth, and extraction conditions to find process efficiencies that lead to cost reductions and/or better yields.

### 2. Description of the Related Art

The term cannabis or “cannabis biomass” encompasses the Cannabis sativa plant and also variants thereof, including subspecies sativa, indica and ruderalis, cannabis strains (also called cultivars), and cannabis chemovars (varieties characterised by chemical composition), which naturally contain different amounts of the individual cannabinoids, and also plants which are the result of genetic crosses. The term “cannabis biomass” is to be interpreted accordingly as encompassing plant material derived from one or more cannabis plants

Cannabis biomass contains a unique class of terpeno-phenolic compounds known as cannabinoids or phytocannabinoids that have been extensively studied since the discovery of the chemical structure of tetrahydrocannabinol (Δ9-THC), commonly known as THC. THC is the main constituent responsible for psychoactive effects. Cannabidiol (CBD) is the primary non-psychoactive cannabinoid and is widely known to have therapeutic potential for a variety of medical conditions. The proportion of cannabinoids in the plant may vary from species to species, and from strain to strain, as well as vary within the same species and strains at different times and seasons. Similarly, the cannabis plant may contain a plurality of terpene, terpenoid or phenolic compounds which may impart their own therapeutic or organoleptic properties to the plant, or may act synergistically with cannabinoids and other components to provide certain effects by what is commonly referred to as the “entourage effect”.

Historical delivery methods involve smoking (combusting) the dried cannabis plant material. Smoking results in adverse effects on the respiratory system via the production of potentially toxic substances and also delivers a variable mixture of active and inactive substances, many of which may be undesirable. Alternative delivery methods such as vaporizing or ingesting typically require extracts of the cannabis biomass (also known as cannabis concentrates or cannabis oils). Raw cannabis biomass may also be more susceptible to possible biological contaminants such as fungi and bacteria than extracts. Often cannabis is grown by “growers” and cannabis extracts produced by “extractors”. In some cases, the growers and extractors may be the same entity.

Cannabis extracts may be obtained using a number of methods, including but not limited to supercritical fluid extraction, solvent extraction of microwave-assisted extraction. In most cases, the extraction efficiency (% recovery of available cannabinoids) and yield of cannabis extract obtained will depend upon the composition of the cannabis biomass used for the extraction, including for example the potency or concentration of cannabinoids present in the cannabis biomass. In some cases, the yield of cannabis extract and the quality of cannabis extract may be different depending on the extraction conditions used to obtain the extract (e.g., solvent type, ratio of solvent to biomass, temperature and time of extraction, etc.). The quality of the cannabis extract may be defined by the potency or concentration of cannabinoids in the extract, or the cannabinoid profile in the extract (e.g., the relative concentrations of various cannabinoids present), or the terpene profile in the extract, relative concentrations of various terpenes present).

The extraction process could be more profitable if the optimal cannabis strain for the specific extractor operation could be used. Those higher margins could be passed onto the grower as an incentive to develop the optimal strains and keep consistent environment conditions for the grow process so that the extraction process is optimal (highest yield and efficiency or optimal mix of cannabinoids and/or other compounds such as terpenes).

Cannabis plants from the same cannabis strain can grow, yield, and look different depending on the growing environment and growing conditions. The number of cannabis strains continues to increase as plants are bred to optimize multiple specific traits (e.g. yield, hardiness, flavor, etc.). It is in the best interest of the strainer, grower and extractor to collect and correlate straining, grow, and extraction information so that the best cannabis strains (i.e. higher yield and efficiency and extract profile) can be developed on a case by case basis (e.g. for each individual grower/extractor interaction).

### SUMMARY OF THE CLAIMED INVENTION

Embodiments of the present invention allows for development of extraction-based analytics to guide upstream decisions (e.g., plant breeding, growing condition). Additionally, growers and extractors of biomass may be made aware of quality tracking and analysis of the biomass product by correlating plant strains and growing conditions with efficiency in the biomass extraction. Such correlations may potentially lead to processing cost reduction, improved product quality and consistency/predictability, and other supply chain efficiencies.

### BRIEF DESCRIPTIONS OF THE DRAWINGS

FIG. 1 illustrates an exemplary network environment in which a system for extraction-based strain engineering may be implemented.

FIG. 2 is a flowchart illustrating an exemplary method for correlating extraction data to strain data.

FIG. 3 is a flowchart illustrating an exemplary method for analyzing correlation data.

FIG. 4 is a flowchart illustrating an exemplary method for correlation-based pricing.

FIG. 5 illustrates an exemplary grower database.

FIG. 6 is a flowchart illustrating an exemplary method for correlation-based grow optimization.

FIG. 7 is a flowchart illustrating an exemplary method for price-based grow recommendation.

### DETAILED DESCRIPTION

Embodiments of the present invention include systems and methods for extraction-based strain engineering. Extraction process may be monitored through the use of sensors, and such sensor data may be correlated to extraction results (e.g., amount and quality of concentrate) with specific strains and growing conditions. Such correlation may further be used to improve the efficiency of the plant breeder and grower process by way of feedback regarding what strains and grow conditions are correlated to high quality or high-efficiency extraction results.

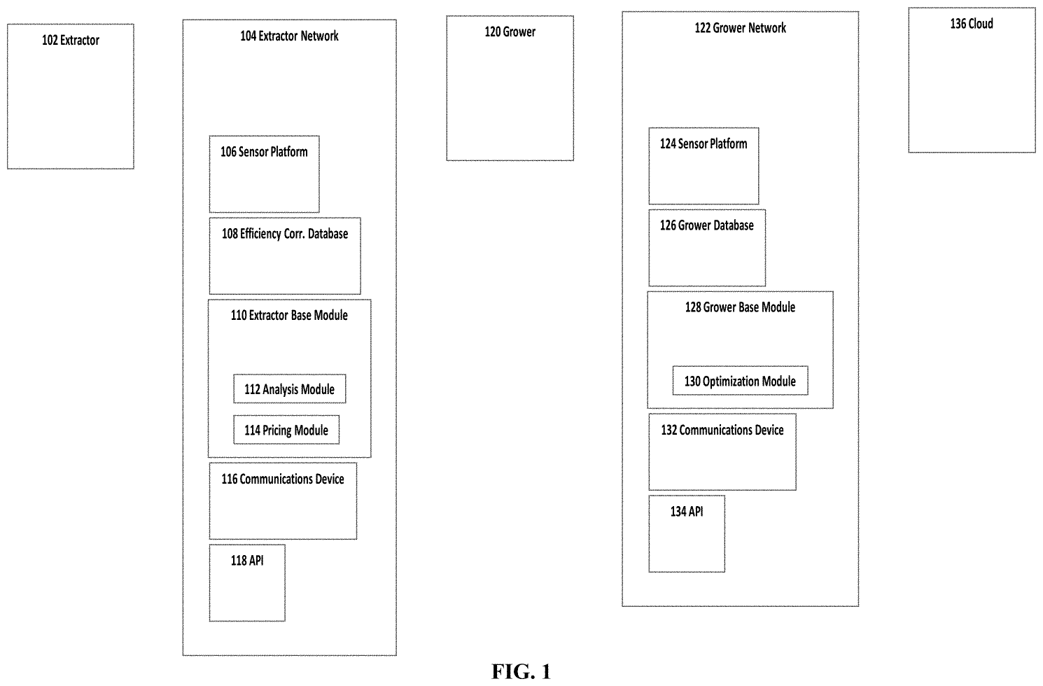

FIG. 1 illustrates an exemplary network environment in which a system for extraction-based strain engineering may be implemented. As illustrated, such network environment may include an extractor device 102, extractor network server 104 (including sensor platform 106, efficiency correlation database 108, extractor base module 110, analysis module 112, pricing module 114, communications device 116, extractor application programming interface (API) 118), grower device 120, grower network 122 (including sensor platform 124, grower database 126, grower base module 128, optimization module 130, communication device 132, grower API 134) and cloud 136.

The extractor device 102 may be associated with any entity that extracts cannabis concentrates from various types of cannabis biomass. Such extractor devices 102 may be monitored to evaluate various properties of the resulting extract.

Extractor device 102 may be associated with a grower network server system 104 that correlates the efficiency of the extraction process with the types of strains and growing conditions of the cannabis biomass, which may thereafter be used to incentivize growers to grow and design cannabis strains optimal to the extractor operations. Such incentives may be implemented by providing feedback to the grower regarding efficiencies of the extraction process in relation to certain plant strains and growth conditions thereof. Strains or conditions that are highly correlated to efficient extraction and high prices ($/kg), for example, may be associated with higher prices for the grower as well.

Sensor platform 106 may contains multiple and different sensors positioned throughout the extraction process performed by extractor device 102. Some of these sensors may be optical sensors, including hyperspectral cameras, imaging sensors), timers, light sensors, etc. Other sensors may perform chemical analyses of the extracts produced by the extraction process performed by extractor device 102. Such sensor data may be indicative of cannabinoid profile, terpene profile, and characterizations of the extraction yield and efficiency.

Efficiency correlation database 108 may store data regarding the efficiency of the extraction process upon a current biomass in association with the strain and growth conditions. Updated on a continuous basis, the data tracked by efficiency correlation database 108 may be analyzed to identify specific strains and/or grow conditions thereof that are highly correlated to efficient extraction for each type of cannabis biomass.

Extractor base module 110 may be software that is executable to manages sensor data, accesses data from grower network servers 122, and communicates with the analysis module 112. Additionally, extractor base module 110 may provide correlation and pricing feedback to growers devices 120.

Analysis module 112 may be inclusive of software executable to analyze sensor data, find correlations between the plant grower database and extraction efficiencies, and execute the pricing module 114 to estimate an adjusted price ($/kg) for the plant breeder/grower Pricing module 114 may be inclusive of software executable to estimate the savings that may be had if the optimal cannabis strain and/or plant grow conditions are used for a given extraction process.

Communication device 116 may include hardware capable of transmitting an analog or digital signal over the telephone, other communication wire, or wirelessly within the network environment. Extractor API 118 allows for communication among the devices associated with the extractor network 104.

Grower device 120 may be associated with any entity that grows the cannabis biomass. Grower device 120 may track properties of the cannabis plants grown by the grower or farm, including specific cannabis plant strains, associated grow conditions, and other characteristics of the cannabis plant.

Grower network server 122 may be a network system that allows for engineered cannabis strains to improve extraction efficiency. Such engineering may be based on collected strain data and grow conditions for specific strain biomass and feedback from extractors. Grower network server 122 may also estimate an adjusted price ($/kg) for the extractor based on growing the new engineered strains.

Sensor platform 124 may contain a group of sensors that identifies the cannabis strain and detects growth conditions (e.g., plant deceases, water frequency, plant density, soil acidity, temperature, light condition, humidity, nutrients etc.). The sensors may include imaging sensors and optical sensors capable of hyperspectral analysis or multi-spectral analysis. Such sensor data may indicate a current point in the growth cycle of the cannabis plants, as well as related moisture levels, sunlight levels, etc. Grower database 126 may store and update continuously regarding grow conditions for each cannabis strain as identified by strain identifier.

Grower base module 128 may be inclusive of any software executable to manage plant strain and sensor data, Grower base module 128 may further initiate execution of the optimization module 130 when feedback from extractor network server 104 is received

Optimization module 130 may be inclusive of software executable to determine the specific combination of strains that are correlated with high-efficiency or high quality results in a given extractor operation based on collected data and a cost estimate.

Communication device 132 may be inclusive of hardware capable of transmitting an analog or digital signal over the telephone, other communication wire, or wirelessly within the network environment of FIG. 1. Grower API 134 may be part of the communications device/server that receives data requests and sends responses within the network environment. Cloud 136 may be inclusive of a variety of available communication network including the Internet. Cloud 136 allows for communications between the different communication devices and modules of FIG. 1 to communicate with each other, as well as with devices external to the illustrated network environment.

Table 1 below illustrates an exemplary efficiency correlation database 108. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

TABLE 1

| | | | | Microwave | Solvent to | Residence | | |
|---|---|---|---|---|---|---|---|---|
| Strain | Biomass | Extraction | | Power | Biomass | Time | Extraction | Extract |
| Name | Number | Conditions | Solvent | Density | Ratio (l/kg) | (min) | Efficiency | Quality |
| Kosher | T001 | Continuous Flow | Ethanol | Low | 10 | 5 | 93% | Excellent |
| Kush | | Microwave | | | | | | |
| Kosher | 1002 | Continuous Flow | Pentane | Med | 10 | 5 | 97% | Very |
| Kush | | Microwave | | | | | | Good |
| Kosher | 1104 | Continuous Flow | Ethanol | Low | 12 | 20 | 98% | Excellent |
| Kush | | Microwave | | | | | | |
| Kosher | 1201 | SFE | CO2 | N/A | 50 | 360 | 75% | Good |
| Kush | | | | | | | | |
| Cannatonic | C220 | Continuous Flow | Ethanol | High | 12 | 10 | 94% | Very |
| | | Microwave | | | | | | Good |
| Cannatonic | C301 | Continuous Flow | Ethanol | Low | 12 | 10 | 87% | Excellent |
| | | Microwave | | | | | | |
| Hemp | H001 | Continuous Flow | Ethanol | Med | 12 | 20 | 83% | Good |
| | | Microwave | | | | | | |
| Hemp | H202 | Continuous Flow | Ethanol | Low | 10 | 20 | 71% | Very |
| | | Microwave | | | | | | good |

As illustrated in Table 1, the efficiency correlation database 108 may store a variety of data regarding specific cannabis strains. Exemplary data in efficiency correlation database may include strain name, strain ID, extraction method, extraction conditions, extraction solvent, microwave power density, extraction solvent ratio, residence time, extraction efficiency, and extract quality (e.g., potency, cannabinoid profile, or terpene profile of extract). In an embodiment, the extraction conditions (e.g., temperature, time, solvent, solvent ratio, etc.) may be represented by a code (e.g., “Extraction Condition A”) to protect confidential extraction information.

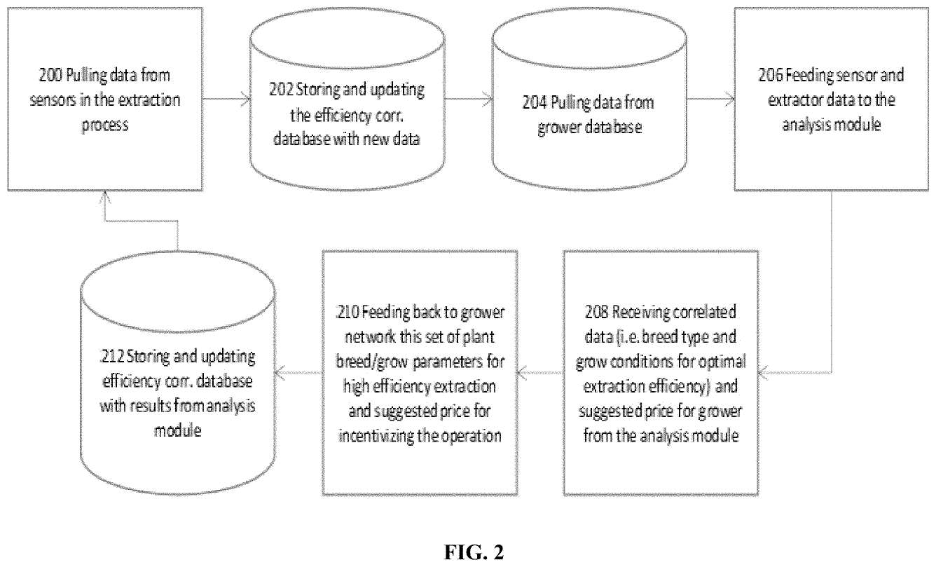

FIG. 2 is a flowchart illustrating an exemplary method for correlating extraction data to strain data. Such method may be performed by execution of the extractor base module 110. The process begins with step 200 in which extractor base module 110 may poll for new sensor events from the extraction process. In step 202, extractor base module 110 may store the new sensor data into the efficiency correlation database 108

In step 204, the new sensor data may trigger the extractor base module 110 to open the communication device 116 and the extractor API 118 in the extractor network 104 to access the grower database 126. In step 206, the extractor base module 110 feeds the sensor data and data from the grower database 126 to the analysis module 112. In step 208, the analysis module 112 sends back to the extractor base module 110 the correlated data (e.g., optimal plant strain/grow conditions for specific extraction operations) and a suggested price for the grower if the grower to supply the identified cannabis plant strain grown under the identified grow conditions.

In step 210, the extractor base module 110 may send feedback to grower device 120 with correlation results and suggested price. In step 212, the extractor base module 110 stores information from analysis module 112 in the efficiency correlation database 108.

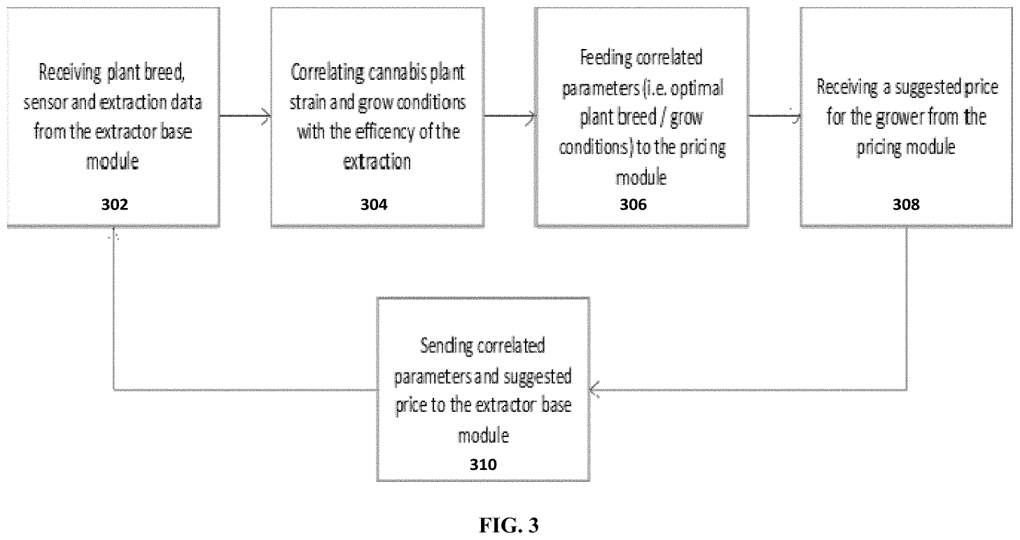

FIG. 3 is a flowchart illustrating an exemplary method for analyzing correlation data. Such method may result from execution of the analysis module 112. The process begins with step 302 in which the extractor base module 110 sends plant strain and sensor data from grower network 122 and extraction data from extractor network 104 to the analysis module 112.

In step 304, the plant parameters (e.g., strain and composition) and grow conditions are correlated to the extraction parameters to find the optimal conditions that give the highest extraction efficiency. For example, the cannabis plant strain may be named: “Kosher Kush,” the biomass number may be “Biomass T104” with an extraction efficiency of 98% and designated “Excellent Extract Quality” (e.g. displaying cannabinoid and terpene profile matching that of the full plant profile).

In step 306, the correlated parameters (e.g., plant strain and efficiency) may be sent to the pricing module 114 to estimate the cost savings to be had if operating at full efficiency under the same constant conditions on a continuous basis. In step 308, the suggested price is received from the pricing module 114, and in step 310, the correlated parameters (e.g., plant strain and efficiency) and suggested price are sent to the extractor base module 110.

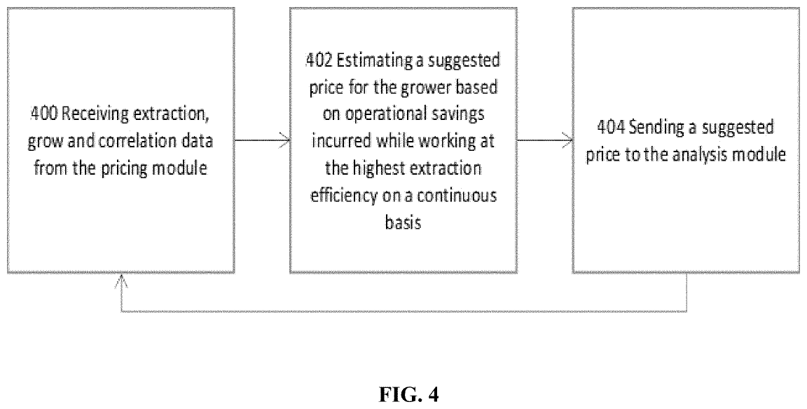

FIG. 4 is a flowchart illustrating an exemplary method for correlation-based pricing. Such method may be performed by execution of the pricing module 114. The process begins with step 400 in which the analysis module 112 feeds extraction, grow and correlation data to the pricing module 114. In step 402, the pricing module 114 calculates the operational savings in using optimal raw materials (plant strain/grow conditions) for the highest extraction efficiency on a continuous basis and estimates a suggested price (in general, higher than the regular price) to be offered to the grower to design the optimal strain. In step 404, the pricing module sends the suggested price for plant breeders/growers to the analysis module 112.

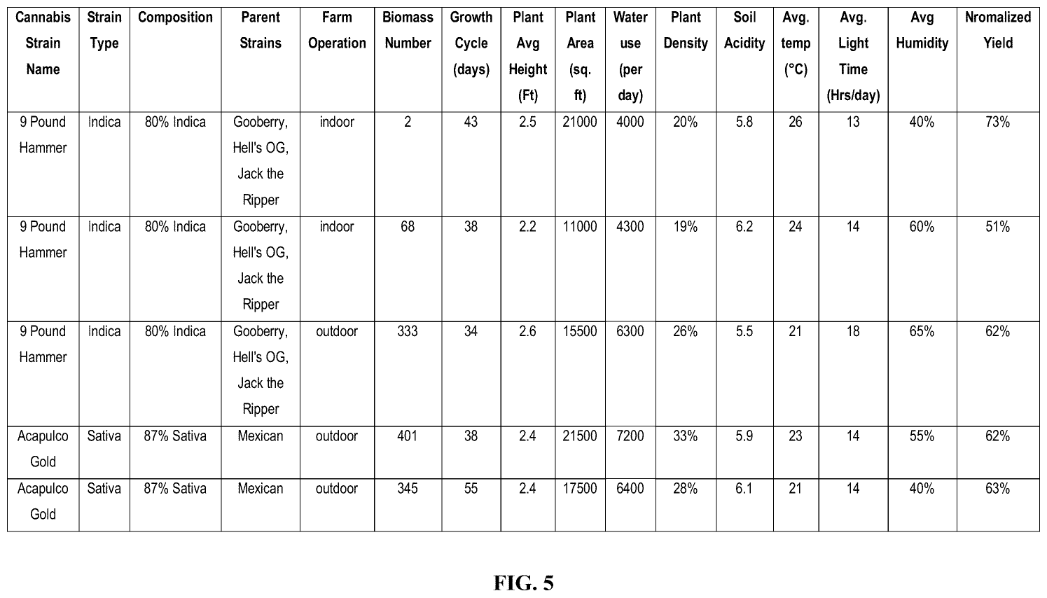

FIG. 5 illustrates an exemplary grower database. Exemplary grow database may store and update continuously the grow conditions/cannabis strain ID. As illustrated, such stored data may include cannabis strain name, strain type, composition, parent strains, farm operation, biomass number, growth cycle days, plant average height, plant area, water use, plant density, soil acidity, average temperature, average light time, average humidity, and normalized yield.

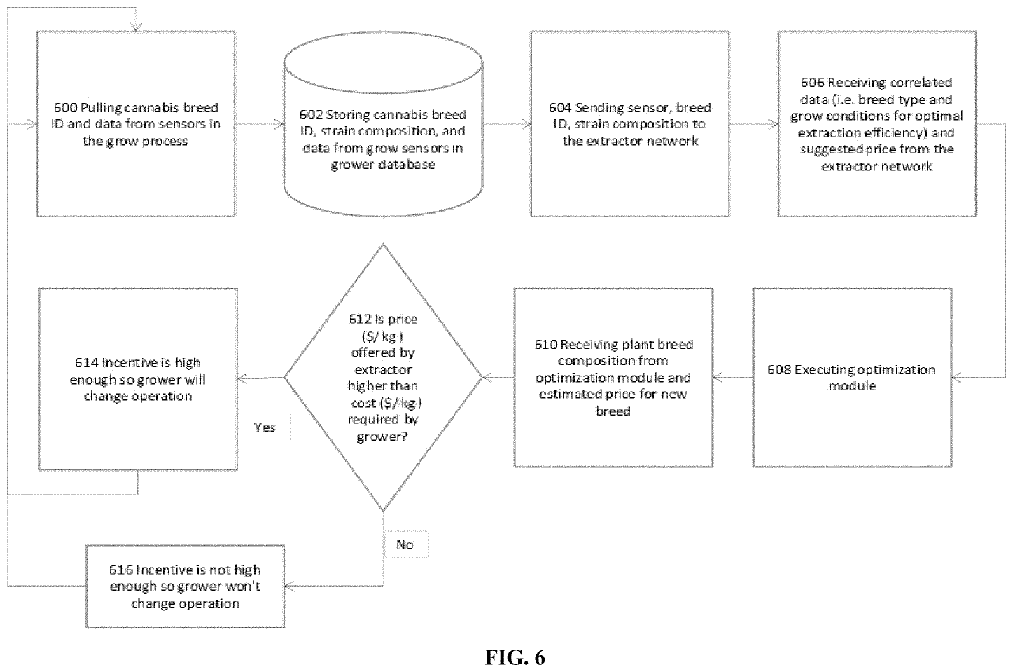

FIG. 6 is a flowchart illustrating an exemplary method for correlation-based grow optimization. Such method may be performed by execution of grower base module 128. The process begins with step 600 in which the receipt of a new plant shipment with the plant strain ID label or new sensor events from the grow process triggers the grower base module 128 to pull new data from sensor and label readers.

In step 602, the new data from sensors (e.g., humidity, plant strain composition and ID) and label readers is stored in the grower database 126. In step 604, new entries in the grower database 126 open the communications with the extractor network 104 and new data is sent to the extractor base module 110. In step 606, correlated data (e.g., strain type and growing conditions) is sent from the extractor network 104 to the grower network 122, so that grower can estimate the cost involved in developing a new cannabis strain and set the farm operations with the optimal grow conditions.

In step 608, the grower base module 128 executes the optimization module 130 to estimate the composition of the new cannabis strain and the cost of creating a new cannabis strain and setting up farm operations in a way that the efficiency operations of a given extractor is optimized. The optimization module 130 may consider inter alia the time and costs associated with creating the new strain.

In step 610, the plant strain composition and the estimated price ($/kg) are received from the optimization module 130. In step 612, it may be determined if the cost is lower than the offered price from the extractor. If yes, the grower is likely to accept the new arrangement in step 614. If not, the method may proceed to step 616 where it may be determined that the incentive is insufficient to justify the operational change.

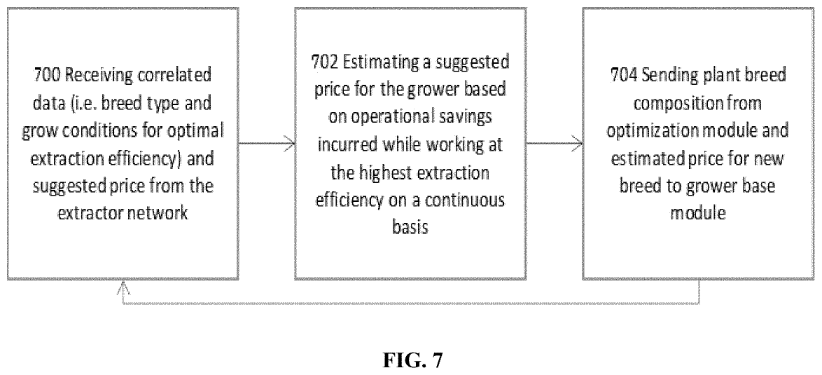

FIG. 7 is a flowchart illustrating an exemplary method for price-based grow recommendation. Such method may be performed by execution of the optimization module 130. The process begins with step 700 in which the grower base module 128 feeds strain data and grow parameters to the optimization module 130.

In step 702, the pricing module 114 estimates the capital investment in developing a new type of strain/the cost to switching strains and perform grow operations under the optimal conditions that the extractor requires. In step 704, the optimization module 130 sends the suggested cost ($/kg) for the specific plant strain needed to the grower base module 128.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology, its practical application, and to enable others skilled in the art to utilize the technology in various embodiments and with various modifications as are suited to the particular use contemplated. It is intended that the scope of the technology be defined by the claims.

What is claimed is:

1. A method for extraction-based strain engineering, the method comprising:
storing grow data regarding a plurality of different types of cannabis plant strains, the grow data including grow conditions for each cannabis plant strain;
evaluating extracts from a plurality of different types of cannabis plant strains, wherein each extract is associated with an extraction efficiency;
selecting one of the extracts based on a comparison of extraction efficiencies among the extracts from the different types of cannabis plant strains;
correlating the extraction efficiency for the selected extract with the associated type of cannabis plant strain and grow conditions; and
generating a recommendation regarding correlated type of cannabis plant strain and grow conditions, wherein the recommendation includes an incentive based on the extraction efficiency.

2. The method of claim 1, wherein evaluating the extracts include collecting sensor data from sensors positioned at different stages of extraction.

3. The method of claim 1, further comprising collecting the grow data from sensors positioned at different stages of growth.

4. The method of claim 3, wherein the collected grow data includes at least one of optical sensor data, hyperspectral data, multi-spectral data, and chemical data.

5. The method of claim 1, further comprising generating a prediction regarding a subsequent biomass of the correlated type of cannabis plant strain, the prediction based on the extraction efficiency of the selected extract.

6. The method of claim 1, wherein evaluating the extracts further identifies at least one of yield, purity, potency, cannabinoid profile, terpene profile, cannabidiol content, and tetrahydrocannabinol content.

7. The method of claim 1, wherein the incentive includes a suggested price.

8. The method of claim 7, wherein the suggested price is based on an increased efficiency of the selected extract in comparison to other extracts.

9. The method of claim 1, wherein the recommendation specifies that the incentive is further based on compliance with the correlated grow conditions.

10. A system for extraction-based strain engineering, the system comprising:
a grow database in memory that stores grow data regarding a plurality of different types of cannabis plant strains, the grow data including grow conditions for each cannabis plant strain; and
an analytics module stored in memory and executable by a processor to:
evaluate extracts from a plurality of different types of cannabis plant strains, wherein each extract is associated with an extraction efficiency;
select one of the extracts based on a comparison of extraction efficiencies among the extracts from the different types of cannabis plant strains;
correlate the extraction efficiency for the selected extract with the associated type of cannabis plant strain and grow conditions; and
generate a recommendation regarding correlated type of cannabis plant strain and grow conditions, wherein the recommendation includes an incentive based on the extraction efficiency.

11. The system of claim 10, wherein the analytics module evaluates the extracts by collecting sensor data from sensors positioned at different stages of extraction.

12. The system of claim 10, further comprising sensors that collect the grow data at different stages of growth.

13. The system of claim 12, wherein the collected grow data includes at least one of optical sensor data, hyperspectral data, multi-spectral data, and chemical data.

14. The system of claim 10, wherein the analytics module is further executable to generate a prediction regarding a subsequent biomass of the correlated type of cannabis plant strain, the prediction based on the extraction efficiency of the selected extract.

15. The system of claim 10, wherein the analytics module evaluates the extracts by identifying at least one of yield, purity, potency, cannabinoid profile, terpene profile, cannabidiol content, and tetrahydrocannabinol content.

16. The system of claim 10, wherein the incentive includes a suggested price.

17. The system of claim 16, wherein the suggested price is based on an increased efficiency of the selected extract in comparison to other extracts.

18. The system of claim 10, wherein the recommendation specifies that the incentive is further based on compliance with the correlated grow conditions.

19. A non-transitory, computer-readable storage medium, having embodied thereon a program executable by a processor to perform a method for extraction-based strain engineering, the method comprising:
storing grow data regarding a plurality of different types of cannabis plant strains, the grow data including grow conditions for each cannabis plant strain;
evaluating extracts from a plurality of different types of cannabis plant strains, wherein each extract is associated with an extraction efficiency;
selecting one of the extracts based on a comparison of extraction efficiencies among the extracts from the different types of cannabis plant strains;
correlating the extraction efficiency for the selected extract with the associated type of cannabis plant strain and grow conditions; and
generating a recommendation regarding correlated type of cannabis plant strain and grow conditions, wherein the recommendation includes an incentive based on the extraction efficiency.
